(19) United States
(12) Patent Application Publication
(10) Pub. No.: US 2025/0288710 A1
(43) Pub. Date: Sep. 18, 2025

(54) OZONE SANITIZING VESSEL RINSER

(71) Applicant: Delta Faucet Company, Indianapolis, IN (US)

(72) Inventors: Kurt Judson Thomas, Indianapolis, IN (US); Michael Scot Rosko, Greenwood, IN (US)

(21) Appl. No.: 19/074,840

(22) Filed: Mar. 10, 2025

Related U.S. Application Data
(60) Provisional application No. 63/565,805, filed on Mar. 15, 2024.

(51) Int. Cl. A61L 2/18; A47L 15/00

(52) U.S. Cl. CPC A61L 2/183; A47L 15/0065; A61L 2202/121; A61L 2202/15; A61L 2202/23

(57) ABSTRACT

A vessel rinsing apparatus operably connected to a source of ozonated fluid and including a drain bowl, a fluid discharge control including trigger, a sprayface having an outlet, and a receptacle that includes a compound, such as activated charcoal, that aids in the degradation and/or decomposition of ozone.

### CROSS-REFERENCE TO RELATED APPLICATION

The present application claims priority to U.S. Provisional Patent Application Ser. No. 63/565,805, filed Mar. 15, 2024, the disclosure of which is expressly incorporated herein by reference.

### BACKGROUND AND SUMMARY OF THE DISCLOSURE

The present invention relates generally to a fluid dispensing system and, more particularly, to a vessel rinsing apparatus for use with containers, such as fluid vessels or receptacles, including cups, bottles and/or glasses in which the rinsing fluid includes ozone (e.g., ozonated water).

Vessel rinsers are known in the art to provide for the cleaning of various containers including rinsers that may be mounted on a sink deck. However, there is a need for a vessel rinser that incorporates treated, such as ozonated, rinsing fluids.

An illustrative vessel rinsing apparatus of the present disclosure includes improvements over prior art glass rinsers. A first illustrative component is an under the sink ozone generator, powered by electrical current, that would generate dissolved ozone in the water stream leading to the vessel rinsing apparatus.

The vessel rinsing apparatus illustratively includes a flow switch to turn on the generator when water is flowing, i.e., the glass was engaged on the vessel rinsing apparatus. Dispensing nozzles may be appropriately sized to give velocity to rinsing streams in the vessel rinsing apparatus at the flow rate achievable by the ozone generator.

A second illustrative component is a receptacle that receives and/or sequesters a compound that degrades and/or decomposes ozone, and that allows the draining water from the rinsed container to pass through it. The receptacle is illustratively formed of a water permeable material, and may be comprised of a layer or mat of porous or woven fiber. Contacting of the ozone containing water with a material that helps to degrade ozone reduces the amount of ozone in the water. The water then drains into the sink to be carried away by the connected plumbing. In some illustrative aspects, the compound that degrades ozone is an ozone decomposing porous material, such as a charcoal, an activated charcoal and/or an activated carbon containing material.

It would be unlikely for ozonated water to bypass the receptacle because if the vessel rinsing apparatus is activated without some sort of vessel over the top of the spray nozzles, water is undesirably sprayed vertically around the sink area. The compound that aids in the degradation of ozone would continue to be effective as long as it was present since it does not rely on surface treatments or coatings to operate. Illustratively, the volume of compound present in the receptacle is configured to exceed any expected consumption by ozone gas over the life span of a faucet.

According to an illustrative embodiment of the present disclosure, a vessel rinsing apparatus includes a hollow, cylindrical mounting shank extending along a longitudinal axis, the mounting shank defining a first fluid passageway. A valve includes a valve seat supported by the mounting shank, a valve stem moveable along the longitudinal axis, and a valve seal supported by the valve stem. The valve stem is moveable between a closed position and an open position. The valve seal is biased against the valve seat in the closed position. A fluid discharge control includes a central body extending along the longitudinal axis and is at least partially disposed within the mounting shank, wherein the central body includes a second fluid passageway. Either or both first and second fluid passageways are operably connected to a source of ozonated fluid. A sprayface is supported by a first end of the central body and includes at least one outlet in fluid communication with the second fluid passageway.

A trigger is operably coupled to the central body, wherein the trigger is axially moveable along the longitudinal axis between a raised position and a lowered position. The source of fluid for the rinse is connected to a source of ozone. Ozone may be added to the rinse by a fluid passageway or shunt between the ozone source and the rinse fluid, optionally by mixing with ozone in a tank that include the rinse fluid. A drain bowl is operably coupled to the mounting shank, the drain bowl being defined by a base, an upwardly extending wall, a surface configured to support a movable vessel, and a receptacle that includes an ozone decomposing porous material, such as an activated carbon containing material (e.g., activated charcoal). The surface includes spaces, for example voids or holes, between individual vanes extending through the surface. The spaces permit the egress of rinse fluid that was in contact with a vessel placed on the surface. The receptacle that houses the compound that aids in the degradation and/or decomposition of ozone is positioned relative to the base such that used rinse fluid drains through at least a portion of the compound before it flows to a drain.

According to a further illustrative embodiment of the present disclosure, a drinkware rinsing apparatus includes a hollow, cylindrical mounting shank extending along a longitudinal axis, the mounting shank defining a first fluid passageway. A valve includes a valve seat supported by the mounting shank, a valve stem moveable along the longitudinal axis, and a valve seal supported by the valve stem. The valve stem is moveable between a closed position and an open position. The valve seal is biased against the valve seat in the closed position. A fluid discharge control includes a central body extending along the longitudinal axis and at least partially disposed within the mounting shank, wherein the central body includes a second fluid passageway. A sprayface is supported by a first end of the central body and includes at least one outlet in fluid communication with the second fluid passageway.

A trigger is operably coupled to the central body, wherein the trigger is axially moveable along the longitudinal axis between a raised position and a lowered position. The trigger is moveable for rotational movement about the longitudinal axis between a first rotation position and a second rotational position. The trigger is biased towards the raised position. Moving the trigger to the lowered position is configured to move the valve seal away from the valve seat causing the valve to be in the open position. A drain bowl is operably coupled to the mounting shank, the drain bowl being defined by a base and an upwardly extending wall. The source of fluid for the rinse is connected to a source of ozone such that the rinse fluid dispensed through the rinsing apparatus may include ozone. The surface of the rinser in contact with the open end of the vessel being rinsed illustratively includes at least one layer of a compound that aids in the degradation and/or decomposition of ozone. The compound illustratively includes an activated carbon containing material, such as activated carbon. In an illustrative embodiment, the rinser is configured such that the ozonated fluid drains through a bed of activated charcoal before it flows to the sink drain.

According to another illustrative embodiment of the present disclosure, a drinkware rinsing apparatus includes a drain bowl defined by a base and an upwardly extending wall. A valve includes a valve seat supported by the mounting shank, a valve stem moveable along the longitudinal axis, and a valve seal supported by the valve stem. The valve stem is moveable between a closed position and an opened position. The valve seal is biased against the valve seat in the closed position. A fluid discharge control includes a central body extending along the longitudinal axis and at least partially disposed within the mounting shank, wherein the central body includes an outlet fluid passageway. A sprayface is supported by a first end of the central body and includes at least one outlet in fluid communication with the fluid passageway. The sprayface is located substantially within the drain bowl.

A trigger is operably coupled to the central body, wherein the trigger is axially moveable along the longitudinal axis between a raised position and a lowered position. The trigger is biased towards the raised position. Moving the trigger to the lowered position is configured to move the valve seal away from the valve seat causing the valve to be in the open position. The rinser is supplied with a source of rinse fluid that always or optionally (i.e., selectively) includes an ozonated fluid such as ozonated water. Discharge of the rinse fluid is through a compound that degrades and/or decomposes ozone. The compound is optionally provided in a receptacle that receives or sequesters the compound while permitting flow-through of the ozonated rinsing fluid. The receptacle that includes the compound may be in the form of a pouch and may have a shape that substantially confirms to the shape of a strainer of the rinser that supports the vessel to be rinsed. The receptacle that includes the compound may be disposed between the upper surface of the strainer of the rinser that engages with the vessel and the drain for spent rinse fluid. In another illustrative embodiment, the receptacle is positioned on the upper surface of strainer. The material used to construct the receptacle may include at least one deformable surface that partially deforms under the weight of vessel positioned on the top of the vessel rinser. The deformation may be sufficient to form at least a partial seal between the opening in the vessel and the partially deformed surface of the receptacle.

According to a further illustrative embodiment of the present disclosure, a drinkware rinsing apparatus includes a hollow, cylindrical mounting shank extending along the longitudinal axis, the mounting shank defining the first fluid passageway. A valve includes a fixed valve member supported within the mounting shank and defining a valve seat, a valve stem moveable along the longitudinal axis, and a valve seal supported by the valve stem. The valve stem is moveable between a closed position and an open position. The valve seal is bias against the valve seat in the closed position. A fluid discharge control includes a central body extending along the longitudinal axis and at least partially disposed within the mounting shank, wherein the central body includes a second fluid passageway. A sprayface is supported by a first end of the central body and includes at least one outlet in fluid communication with the second fluid passageway.

A trigger is operably coupled to the central body. The trigger is axially moveable along the longitudinal axis between a raised position and lowered position. The trigger is bias towards the raised position. Moving the trigger to the lowered position is configured to move the valve seal away from the valve seat causing the valve to be in the open position. A drain bowl is operably coupled to the mounting shank, the drain bowl being defined by a base and an upwardly extending wall. A drain channel illustratively extends through the upwardly extending wall of the drain bowl. In an installed condition of the rinsing apparatus, the drain bowl is positioned above the mounting deck, at least a portion of the drain channel is positioned above the sink basin, and the drain channel is configured to receive fluid from the drain bowl and discharge the fluid above and into the sink basin. The bulk of the discharge fluid flows through a compound that aids in the degradation or decomposition of ozone. In some illustrative embodiments, the compound is received in a receptacle that is permeable to fluid, but impermeable to particles of the compound.

An illustrative embodiment vessel rinsing apparatus includes a hollow, cylindrical mounting shank extending along a longitudinal axis, the mounting shank defining a first fluid passageway. A valve includes a valve seat supported by the mounting shank, a valve stem movable along the longitudinal axis, and a valve seal supported by the valve stem. The valve stem is movable between a closed position and an open position, the valve seal being biased against the valve seat in the closed position. A source of an ozonated fluid is operably connected to first fluid passageway. A fluid discharge control includes a central body extending along the longitudinal axis and at least partially disposed within the mounting shank, wherein the central body includes a second fluid passageway. A sprayface is supported by a first end of the central body and includes at least one outlet in fluid communication with the second fluid passageway. Illustratively, a strainer is configured to receive and support an object placed on the surface of the strainer. A receptacle is illustratively positioned in spaced relation to the strainer, wherein the receptacle includes a porous material helps to degrade or decompose the ozone in the ozonated fluid. A drain bowl includes a first surface disposed towards the strainer, a second surface disposed towards a fluid drain.

An illustrative embodiment vessel rinsing apparatus further includes a trigger operably coupled to the central body, wherein the trigger is axially moveable along the longitudinal axis between a raised position and a lowered position. The trigger is biased toward the raised position. Moving the trigger to the lowered position is configured to move the valve seal away from the valve seat causing the valve to be in the open position.

Another illustrative embodiment vessel rinsing apparatus includes a drain channel in fluid communication with the drain bowl. In an illustrative embodiment, the drain channel extends through the upwardly extending wall of the drain bowl. In an installed condition of the rinsing apparatus, the drain bowl is positioned above a mounting deck, at least a portion of the drain channel is positioned above a sink basin, and the drain channel is configured to receive fluid from the drain bowl and discharge the fluid above and into the sink basin.

According to a further illustrative embodiment vessel rinsing apparatus, the receptacle is substantially impermeable to the flow of the porous material and is substantially permeable to the flow of fluid.

According to another illustrative embodiment vessel rinsing apparatus, the receptacle is positioned below the strainer.

According to a further illustrative embodiment vessel rinsing apparatus, the receptacle is positioned on top of the strainer such that the top of the receptacle is in contact with an objects place on the strainer.

According to another illustrative embodiment vessel rinsing apparatus, the receptacle is comprised of a material that partially deforms to conform to the shape of objects placed on the top surface of the receptacle.

According to a further illustrative embodiment vessel rinsing apparatus, the porous material is charcoal.

According to another illustrative embodiment vessel rinsing apparatus, the charcoal is activated charcoal.

According to a further illustrative embodiment of the present disclosure, a vessel rinsing apparatus includes a drain bowl, a fluid discharge control having a central body supported for movement relative to the drain bowl, and a sprayface supported by the central body and including an outlet. A receptacle includes an ozone decomposition material configured to receive ozonated water discharged from the outlet of the sprayface.

According to another illustrative embodiment of the present disclosure, a vessel rinsing apparatus includes a hollow, cylindrical mounting shank extending along a longitudinal axis, the mounting shank defining a first fluid passageway, and a drain bowl operably coupled to the mounting shank. A valve includes a valve seat supported by the mounting shank, a valve stem moveable along the longitudinal axis, a valve seal supported by the valve stem, and wherein the valve stem is moveable between a closed position and an open position, the valve seal being biased against the valve seat in the closed position. A source of ozonated fluid is operably coupled to the first fluid passageway. A fluid discharge control includes a central body extending along the longitudinal axis and at least partially disposed within the mounting shank, wherein the central body includes a second fluid passageway. A sprayface is supported by a first end of the central body and includes at least one outlet in fluid communication with the second fluid passageway.

Additional features and advantages of the present invention will become apparent to those skilled in the art upon consideration of the following detailed description of illustrative embodiments exemplifying the best mode of carrying out the invention as presently perceived.

### BRIEF DESCRIPTION OF THE DRAWINGS

The foregoing aspects and many of the intended advantages of this invention will become more readily appreciated as the same becomes better understood by reference to the following detailed description of exemplary embodiments when taken in conjunction with the accompanying drawings, wherein:

FIG. 1 is a perspective view of an illustrative vessel rinsing apparatus mounted to a sink deck fluidly coupled to a source of ozonated water;

FIG. 2A is a perspective view of the vessel rinsing apparatus of FIG. 1;

FIG. 2B is a top exploded perspective view of the vessel rinsing apparatus of FIG. 2A;

FIG. 2C is a bottom exploded perspective view of the vessel rinsing apparatus of FIG. 2A;

FIG. 3A is a perspective view of another illustrative embodiment of the vessel rinsing apparatus of FIG. 2A;

FIG. 3B is a top exploded perspective view of the vessel rinsing apparatus of FIG. 3A; and

FIG. 3C is a bottom exploded perspective view of the vessel rinsing apparatus of FIG. 3A.

### DETAILED DESCRIPTION OF THE DRAWINGS

For the purposes of promoting and understanding the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of principles in the invention which would normally occur to one skilled in the art to which the invention relates.

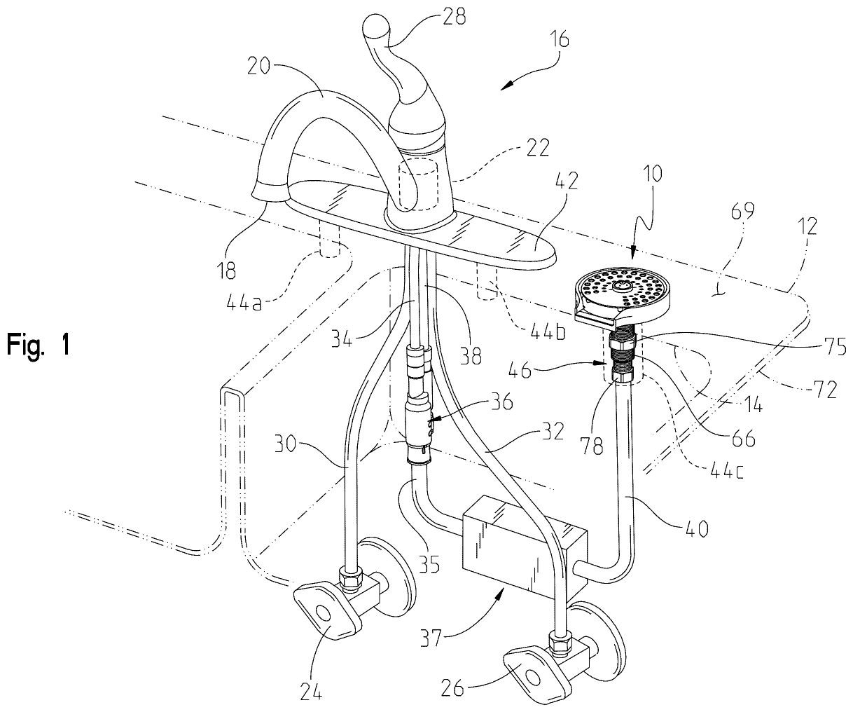

With reference initially to FIG. 1, an illustrative vessel rinsing apparatus or glass rinser 10 is shown supported by a conventional mounting deck, such as a sink deck 12 supporting a sink basin 14. A faucet 16 may be supported by the sink deck 12 for discharging water from a water outlet 18 into the sink basin 14. The faucet 16 may be of conventional design, including a delivery spout 20 defining the water outlet 18, and a manual mixing valve 22 for controlling water flow from a hot water supply 24 and a cold water supply 26 to the water outlet 18. The mixing valve 22 includes a valve handle 28 to control the flow rate and the temperature of water delivered to the water outlet 18. A hot water supply tube 30 may fluidly couple the hot water supply 24 to a first inlet of the mixing valve 22, and a cold water supply tube 32 may fluidly couple the cold water supply 26 to a second inlet of the mixing valve 22. A mixed water outlet tube 34 may fluidly couple an outlet of the mixing valve 22 to a diverter valve 36.

The diverter valve 36 illustratively controls (e.g., toggles) water flow between the outlet 18 of the delivery spout 20 and the vessel rinsing apparatus 10. An illustrative diverter valve 36 is detailed in U.S. Pat. No. 8,646,476 to Thomas et al., the disclosure of which is expressly incorporated by herein by reference. A spout outlet tube 38 fluidly couples the diverter valve 36 to the outlet 18, a dispenser tube 35 fluidly couples the diverter valve 36 to a water treatment device, illustratively an ozonated fluid generator or ozone generator 37. A dispenser tube 40 fluidly couples the ozonated fluid generator 37 to the vessel rinsing apparatus 10. An illustrative ozone generator may of the type detailed in U.S. Patent Application Publication No. 2021/0179461 to Rosko et al., the disclosure of which is expressly incorporated herein by reference.

The mixing valve 22 may be manipulated by the handle 28 to control the temperature and the flow rate of water delivered to the active outlet(s) selected by the diverter valve 36 (e.g., the delivery spout outlet 18 and the glass rinsing apparatus 10). The various water tubes 30, 32, 34, 35, 38 and 40 may be formed of a flexible polymer, such as a cross-linked poly-ethylene (PEX).

An escutcheon 42 may be positioned intermediate the sink deck 12 and the delivery spout 20. A plurality of mounting apertures 44 may extend within the sink deck 12, wherein at least some of the apertures 44a, 44b may be used to couple the faucet 16 to the sink deck 12. In the illustrative embodiment, the glass rinsing apparatus 10 is fixed to the mounting aperture 44c that would conventionally support a kitchen side sprayer (not shown) adjacent the sink basin 14. Such positioning facilitates draining of the vessel rinsing apparatus 10 into the sink basin 14.

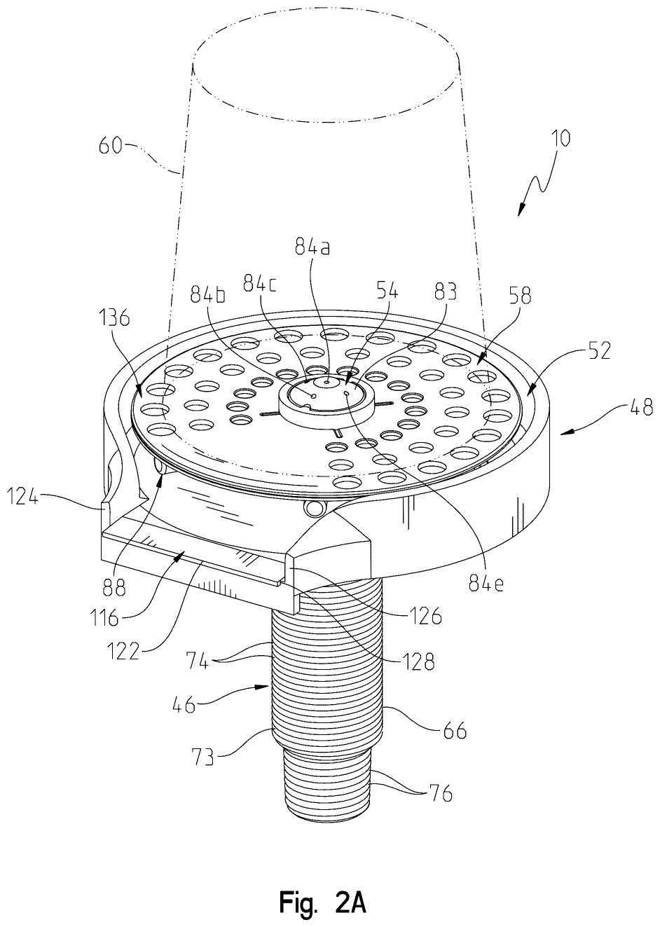

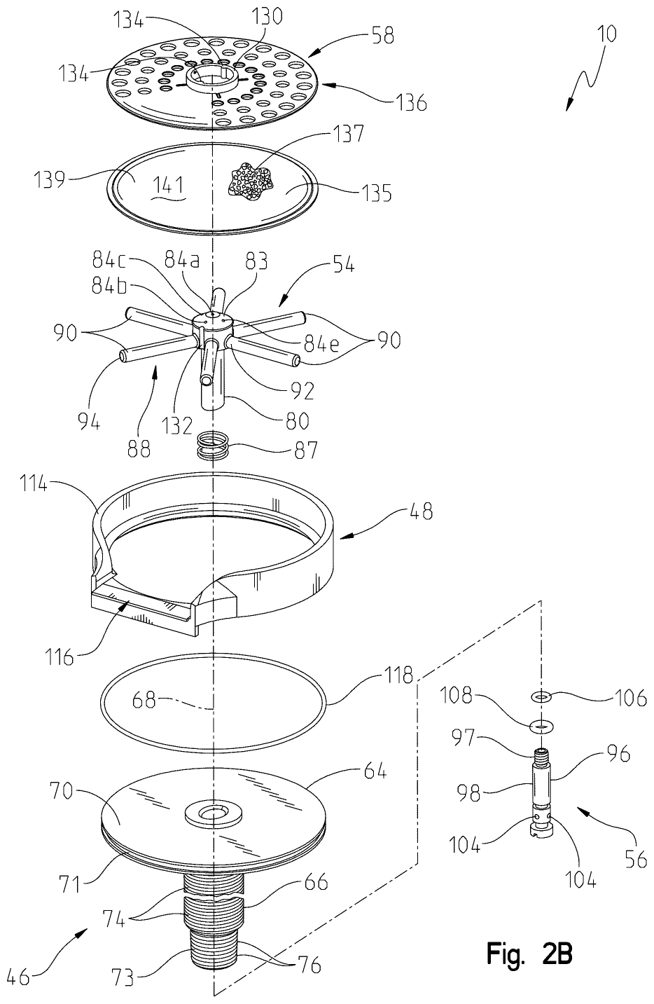

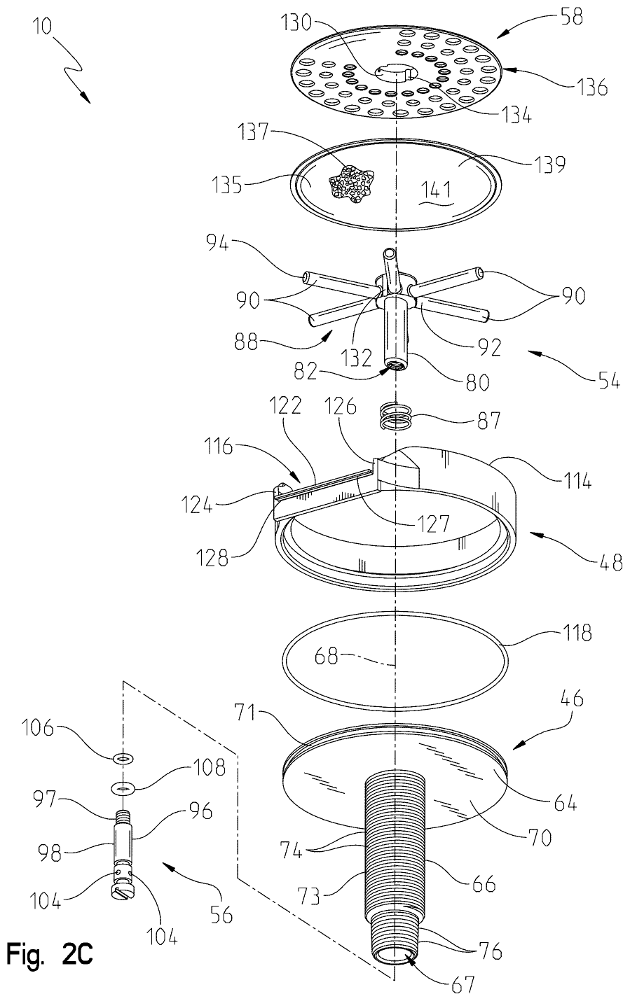

With reference to FIGS. 2A-2C, the illustrative vessel rinsing apparatus 10 includes a mounting base 46 configured to be coupled to the sink deck 12. An escutcheon 48 illustratively cooperates with the mounting base 46 to define a contoured drain bowl 52 for the collection and disposal of debris and wastewater. A fluid discharge member 54 is operably coupled to the mounting base 46 and to a valve member 56. Illustratively, a trigger plate 58 is supported by the fluid discharge member 54 and is configured to be engaged by a vessel 60 (e.g., a cup, glass or other container) for activating water flow through the fluid discharge member 54 and into an interior of the vessel 60.

The mounting base 46 illustratively includes an upper coupler, such as an upper plate 64, and a downwardly extending mounting shank 66 defining an opening 67 extending along a longitudinal axis 68. The upper plate 64 is configured to be supported above an upper surface 69 of the sink deck 12 and illustratively comprises a disk 70 having a circular peripheral edge 71. The mounting shank 66 is configured to extend through one of the mounting apertures 44c formed within the sink deck 12 and below a lower surface 72 of the sink deck 12. The mounting shank 66 illustratively includes a stepped cylindrical tube 73 having upper external threads 74 configured to threadably couple with a mounting nut 75 for securing the mounting base 46 to the sink deck 12 (FIG. 1). Illustratively, the dispenser tube 40 is fluidly coupled to the mounting shank 66 to provide water from a water supply (illustratively via the mixing valve 22 through the diverter valve 36). More particularly, the cylindrical tube 73 of the mounting shank 66 illustratively includes lower external threads 76 configured to engage with a fitting 78 to fluidly couple the dispenser tube 40 to the mounting shank 66 (FIG. 1).

With reference to FIGS. 2B and 2C, the fluid discharge member 54 cooperates with the mounting base 50 to deliver water from the dispenser tube 40. The fluid discharge member 54 illustratively includes a central body 80 defining a fluid passageway 82 and having a sprayface 83 supporting a plurality of nozzles 84 in fluid communication with the fluid passageway 82. More particularly, the nozzles 84 may include a center nozzle 84a and a plurality of peripheral nozzles 84b, 84c, 84d, 84e positioned radially outward from the center nozzle 84a. It should be appreciated that the number and placement of the nozzles 84 may vary.

In one illustrative embodiment, the nozzles 84 may be oriented to direct water in different desired positions within an interior of the vessel. For example, the center nozzle 84a may be configured to discharge water upwardly parallel to the longitudinal axis 68. Illustratively, a first peripheral nozzle 84b may be oriented at a first angle from the longitudinal axis 68 for directing water to a distal corner of a mug, a second peripheral nozzle 84c may be oriented at a second angle from the longitudinal axis 68 for directing water to a distal corner of a highball glass, a third peripheral nozzle 84d may be oriented at a third angle from the longitudinal axis 68 for directing water to a distal corner of a tumbler or pint glass, and a forth peripheral nozzle 84e may be oriented at a fourth angle from the longitudinal axis 68 for directing water to a distal portion of a wine glass.

The central body 80 of the fluid discharge member 54 is slideably received within the opening 67 of the mounting shank 66 such that it is movable along the longitudinal axis 68. More particularly, the fluid discharge member 54 is configured to move along the longitudinal axis 68 between a raised (or rest) position, and a lowered (or active) position. A spring 87 may cooperate with the mounting base 46 to bias the discharge member 54 to the upper position. The fluid discharge member 54 may be restrained from rotating relative to the mounting base 50. Illustratively, the central body 80 has an elongated cross section (e.g., oval) thereby preventing rotation within the opening 67. Other rotatable couplers, such as a key received within a keyway, may be substituted therefor.

A trigger 88 extends outwardly from the central body 80. The trigger 88 illustratively includes a plurality of radially outwardly extending vanes 90, such as arms or posts, each having a first end 92 supported by the central body 80 and a freely supported second end 94. The arms 90 are radially spaced apart from each other to define spaces or openings therebetween.

The valve member 56 illustratively comprises a valve pin or shaft 96 operably coupled to the fluid discharge member 54 (e.g., via a threaded coupling 97), and is configured to move with the trigger 88 to control water flow through the central body 80 to the plurality of nozzles 84.

More particularly, the valve shaft 96 illustratively includes a hollow body 98 defining a fluid passageway 102 in fluid communication with the fluid passageway 82 of the central body 80 via radial openings 104. Upper and lower o-rings 106 and 108 are positioned above and below openings 104, respectively. The valve member 56 may be a separate component from the fluid discharge member 54 or formed integral therewith.

Referring further to FIGS. 2B and 2C, a removable trigger plate 58 may comprise a strainer 136 including a base 138 and a plurality of circumferentially spaced drain openings 140 arranged in a plurality of radially spaced rows. A receptacle 135 filled with an ozone decomposing porous material, such as activated charcoal or activated carbon containing material 137, is illustratively positioned on the upper surface of strainer 136. Optionally, the rim of a vessel 60 contacts the upper surface of receptacle 135.

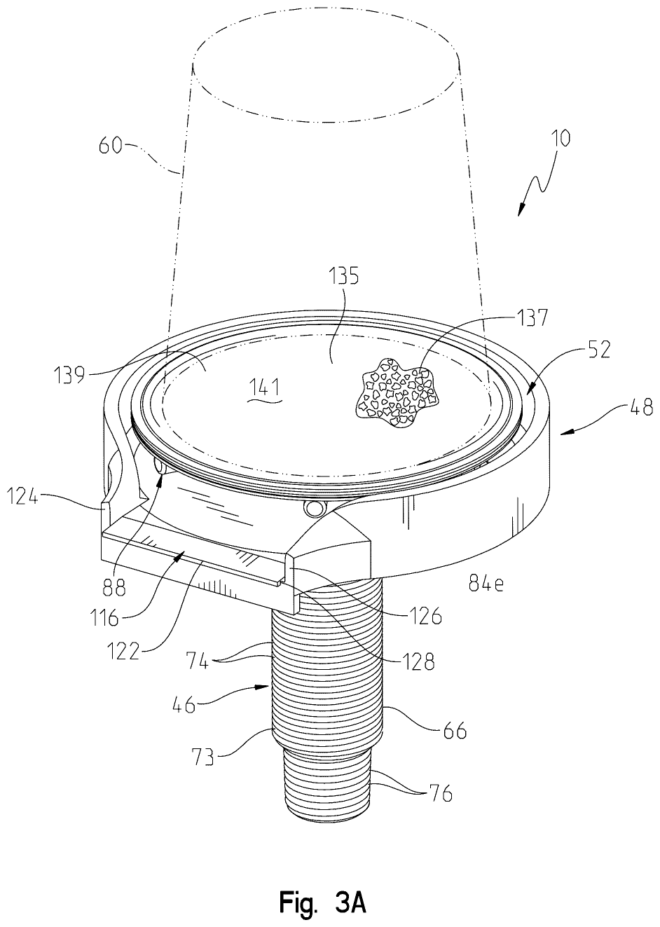

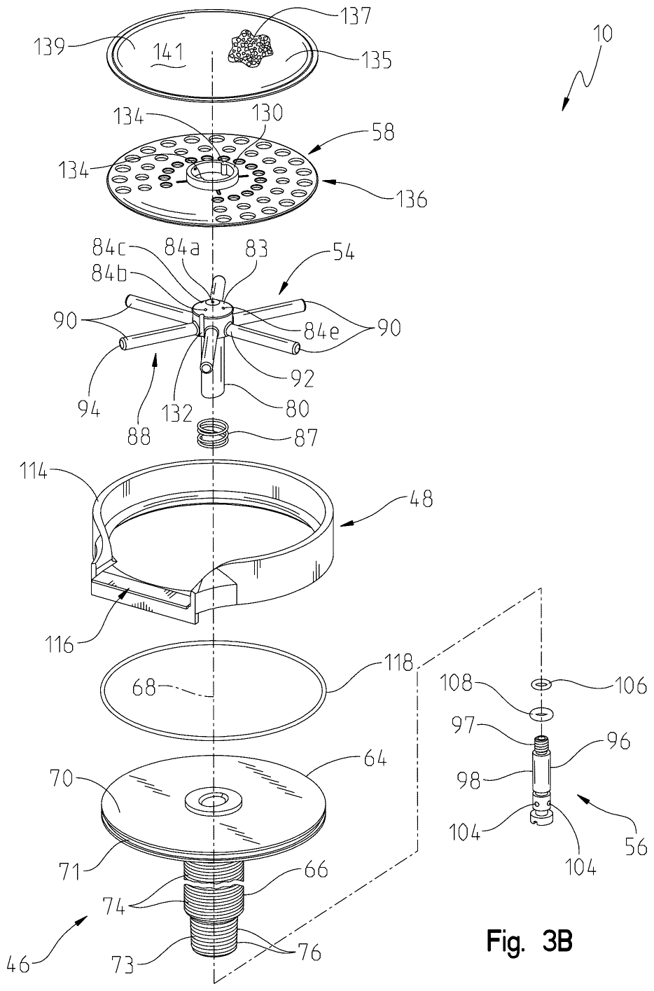

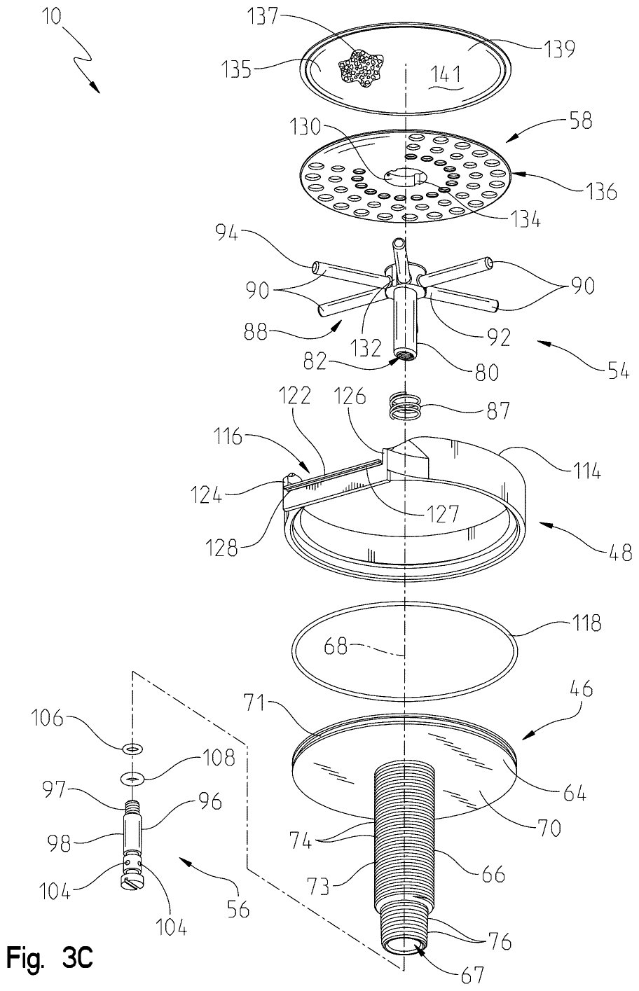

Referring to FIGS. 3B and 3C, the removable trigger plate 58 may comprise a strainer 136 including a base 138 and a plurality of circumferentially spaced drain openings 140 arranged in a plurality of radially spaced rows. The activated charcoal receptacle 135 filed with ozone decomposing porous material, such as activated charcoal or activated carbon containing material 137, so as to contact the lower surface of strainer 136. Optionally, the rim of a vessel 60 contacts the upper surface of strainer 136.

The receptacle 135 illustratively includes an outer membrane 139 receiving the activated charcoal 137. The membrane 139 may be a filter material that is permeable to water but impermeable to particles of activated charcoal 137. More particularly, the membrane 139 may be a layer or mat of porous or woven fiber receiving the activated charcoal 137. The outer surface 141 of the receptacle 135 is deformable for at least partial sealing interaction with a vessel in contact therewith.

Although the invention has been described in detail with reference to certain preferred embodiments, variations and modifications exist within the spirit and scope of the invention as described and defined in the following claims.

What is claimed is:

1. A vessel rinsing apparatus comprising:
a hollow, cylindrical mounting shank extending along a longitudinal axis, the mounting shank defining a first fluid passageway;
a valve including:
a valve seat supported by the mounting shank,
a valve stem movable along the longitudinal axis,
a valve seal supported by the valve stem, and
wherein the valve stem is movable between a closed position and an open position, the valve seal being biased against the valve seat in the closed position;
a source of an ozonated fluid operably connected to first fluid passageway;
a fluid discharge control including:
a central body extending along the longitudinal axis and at least partially disposed within the mounting shank, wherein the central body includes a second fluid passageway, and
a sprayface supported by a first end of the central body and including at least one outlet in fluid communication with the second fluid passageway;
a drain bowl operably coupled to the mounting shank; and
a receptacle positioned in fluid communication with the second fluid passageway, wherein the receptacle includes a porous material configured to degrade ozone in the ozonated fluid.

2. The vessel rinsing apparatus of claim 1, further comprising:
a strainer operably coupled to the central body and configured to support an object; and
wherein the receptacle is positioned below or above the strainer.

3. The vessel rinsing apparatus of claim 2, wherein the receptacle is positioned on top of the strainer such that the top of the receptacle is in contact with an object placed on the strainer.

4. The vessel rinsing apparatus of claim 1, further including;
a trigger operably coupled to the central body,
wherein the trigger is axially moveable along the longitudinal axis between a raised position and a lowered position,
wherein the trigger is biased toward the raised position, and
wherein moving the trigger to the lowered position is configured to move the valve seal away from the valve seat causing the valve to be in the open position.

5. The vessel rinsing apparatus of claim 1, further comprising:
a drain channel fluidly coupled to the drain bowl; and
wherein in an installed condition of the rinsing apparatus, the drain bowl is positioned above the mounting deck, at least a portion of the drain channel is positioned above the sink basin, and the drain channel is configured to receive fluid from the drain bowl and discharge the fluid above and into the sink basin.

6. The vessel rinsing apparatus of claim 1, wherein the receptacle is substantially impermeable to the flow of the porous material and is substantially permeable to the flow of fluid.

7. The vessel rinsing apparatus of claim 6, wherein the receptacle is comprised of a material that partially deforms to conform to the shape of objects placed on a top surface of the receptacle.

8. The vessel rinsing apparatus of claim 1 wherein the porous material is an activated carbon containing material.

9. A vessel rinsing apparatus comprising:
a drain bowl;
a fluid discharge control including:
a central body supported for movement relative to the drain bowl, and
a sprayface supported by the central body and including an outlet; and
a receptacle including an ozone decomposition material configured to receive ozonated water discharged from the outlet of the sprayface.

10. The vessel rinsing apparatus of claim 9, further comprising a source of ozonated water fluidly coupled to the fluid discharge control.

11. The vessel rinsing apparatus of claim 10, further comprising:
a hollow, cylindrical mounting shank extending along a longitudinal axis, the mounting shank defining a first fluid passageway;
a valve including:
a valve seat supported by the mounting shank,
a valve stem movable along the longitudinal axis,
a valve seal supported by the valve stem,
wherein the valve stem is movable between a closed position and an open position, the valve seal being biased against the valve seat in the closed position; and
wherein the source of ozonated water is fluidly coupled to first fluid passageway.

12. The vessel rinsing apparatus of claim 11, wherein:
the central body extends along the longitudinal axis and is at least partially disposed within the mounting shank, the central body including a second fluid passageway; and
the sprayface is supported by a first end of the central body and the outlet is in fluid communication with the second fluid passageway.

13. The vessel rinsing apparatus of claim 12, further including;
a trigger operably coupled to the central body,
wherein the trigger is axially moveable along the longitudinal axis between a raised position and a lowered position,
wherein the trigger is biased toward the raised position, and
wherein moving the trigger to the lowered position is configured to move the valve seal away from the valve seat causing the valve to be in the open position.

14. The vessel rinsing apparatus of claim 9, further comprising:
a strainer operably coupled to the central body and configured to support an object; and
wherein the receptacle is positioned on top of the strainer such that the top of the receptacle is in contact with an object placed on the strainer.

15. The vessel rinsing apparatus of claim 9, further comprising:
a drain channel fluidly coupled to the drain bowl; and
wherein in an installed condition of the rinsing apparatus, the drain bowl is positioned above the mounting deck, at least a portion of the drain channel is positioned above the sink basin, and the drain channel is configured to receive fluid from the drain bowl and discharge the fluid above and into the sink basin.

16. The vessel rinsing apparatus of claim 9, wherein the receptacle is substantially impermeable to the flow of the porous material and is substantially permeable to the flow of fluid.

17. The vessel rinsing apparatus of claim 16, wherein the receptacle is comprised of a material that partially deforms to conform to the shape of objects placed on a top surface of the receptacle.

18. The vessel rinsing apparatus of claim 9, wherein the porous material is an activated carbon containing material.

19. A vessel rinsing apparatus comprising:
a hollow, cylindrical mounting shank extending along a longitudinal axis, the mounting shank defining a first fluid passageway;
a drain bowl operably coupled to the mounting shank;
a valve including:
a valve seat supported by the mounting shank,
a valve stem movable along the longitudinal axis,
a valve seal supported by the valve stem, and
wherein the valve stem is movable between a closed position and an open position, the valve seal being biased against the valve seat in the closed position;
a source of an ozonated fluid operably connected to first fluid passageway; and
a fluid discharge control including:
a central body extending along the longitudinal axis and at least partially disposed within the mounting shank, wherein the central body includes a second fluid passageway, and
a sprayface supported by a first end of the central body and including at least one outlet in fluid communication with the second fluid passageway.

20. The vessel rinsing apparatus of claim 19, further comprising a receptacle positioned in fluid communication with the second fluid passageway, wherein the receptacle includes a porous material configured to degrade ozone in the ozonated fluid.
